(19) United States
(12) Patent Application Publication
(10) Pub. No.: US 2016/0176803 A1
(43) Pub. Date: Jun. 23, 2016

(54) PROCESS FOR THE ETHERIFICATION OF AMINO ALCOHOLS AT LOW TEMPERATURES

(71) Applicant: BASF SE, Ludwigshafen am Rhein (DE)

(72) Inventors: Melanie WEINGARTEN, Ratzeburg (DE); Wolfgang Siegel, Limburgerhof (DE)

(73) Assignee: BASF SE, Ludwigshafen am Rhein (DE)

(21) Appl. No.: 14/962,484

(22) Filed: Dec. 8, 2015

(30) Foreign Application Priority Data
Dec. 18, 2014 (EP) 14198751.1

(51) Int. Cl. C07C 213/06

(52) U.S. Cl. CPC C07C 213/06

(57) ABSTRACT

A process for the preparation of the ether of formula I

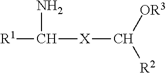

from an amino alcohol of formula II

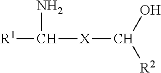

where R₁ and R₂ independently from one another are hydrogen or an alkyl group with 1 to 10 C atoms, R₃ is an alkyl group with 1 to 10 carbon atoms and
X is a bond or a hydrocarbon group with 1 to 10 carbon atoms
comprising
a) protecting the primary amino group in formula II with a protecting agent
b) deprotonating the protected amino alcohol obtained in step a) with a deprotonating agent
c) alkylation of the anion obtained in step b) with an alkylation agent to give the corresponding ether and finally
d) removal of the protecting group getting back the primary amino group and giving the ether of formula I,
wherein process step b) is performed at a temperature of at maximum 120° C. and process step c) is performed at a temperature of at maximum 80° C.

The present invention relates to a process for the preparation of the ether of formula I

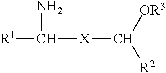

from an amino alcohol of formula II

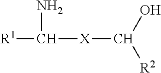

where R₁ and R₂ independently from one another are hydrogen or an alkyl group with 1 to 10 C atoms, R₃ is an alkyl group with 1 to 10 carbon atoms and
X is a bond or a hydrocarbon group with 1 to 10 carbon atoms
comprising
a) protecting the primary amino group in formula II with a protecting agent
b) deprotonating the protected amino alcohol obtained in step a) with a deprotonating agent
c) alkylation of the anion obtained in step b) with an alkylation agent to give the corresponding ether and finally
d) removal of the protecting group getting back the primary amino group and giving the ether of formula I,
wherein process step b) is performed at a temperature of at maximum 120° C. and process step c) is performed at a temperature of at maximum 80° C.

Ethers of formula I are chemical intermediates which are, for example, used for the synthesis of pharmaceuticals, plant protecting agents as herbicides, insecticides or fungicides.

Compounds of formula I may be obtained by etherification of amino alcohols. As such amino alcohols have two functional groups (a primary amino group and a hydroxy group) a selective etherification of the hydroxy group becomes problematic. Mono- or di-alkylation of the nitrogen atom will occur sometimes even preferentially. In particular the by-product with a mono-alkylated nitrogen has to be avoided as the boiling point of such by-product is quite similar to the boiling point of the desired ether of formula I. Hence any separation of such by-product by distillation becomes difficult.

In addition it is often required that the compound of formula I is a defined stereo isomer. Hence an amino alcohol with a defined stereo isomerism is selected as starting material, for example a pure (S) or (R) isomer. Any isomerization during the preparation of the ether has to be avoided and the ether obtained should finally have the same stereo isomerism as the amino alcohol.

DE-A 103 44 447 discloses a process for the etherification of amino alcohols having an unsubstituted amino group and a hydroxyl group. The process described is a two-step process. In a first step an alkali alcoholate is used to prepare the alcoholate of the amino alcohol. In a second step the alcoholate of the amino alcohol is alkylated with an alkylating agent to finally form the corresponding ether.

The use of protecting groups in organic synthesis is generally known. In Tetrahedron Vol. 44, No 17, pp 5495 to 5506 (1988) the use of Schiff bases as protecting groups in the synthesis of amino acids is described by Lucette Duhamel et al.

It is the object of the present invention to improve the process for the preparation of ethers of formula I. The improved process should be very effective and easy to perform. The yield of amino alcohol ethers should be high and any by-products, specifically by-products with substitution at the nitrogen should be avoided. The overall selectivity of the ethers and the retention of the stereo chemistry should be as high as possible.

Accordingly, a process as defined above has been found.

To the Ether for Formula I

The process claimed is a process for the preparation of ether of formula I

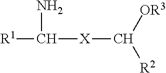

from an amino alcohol of formula II

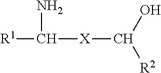

where R₁ and R₂ independently from one another are hydrogen or an alkyl group with 1 to 10 C atoms, R₃ is an alkyl group with 1 to 10 carbon atoms and X is a bond or a hydrocarbon group with 1 to 10 carbon atoms.

Preferably R₁ and R₂ independently from each other are hydrogen or an alkyl group with 1 to 4 C atoms.

Preferably R₃ in formula I is an alkyl group with 1 to 4 C atoms.

Preferably X is a bond or an alkylene group with 1 to 10 carbon atoms, most preferably X is a bond.

In a particularly preferred embodiment the compound of formula I is a compound wherein R₁ is hydrogen or an alkyl group with 1 to 4 C atoms, R₂ is hydrogen, R₃ is an alkyl group with 1 to 4 C atoms and X is a bond or an alkylene group with 1 to 10 carbon atoms;

correspondingly compound of formula II is a compound wherein R₁ is hydrogen or an alkyl group with 1 to 4 C atoms, R₂ is hydrogen and X is a bond or an alkylene group with 1 to 10 carbon atoms.

In a most preferred embodiment the compound of formula I is a compound wherein R₁ is a methyl group, R₂ is hydrogen, R₃ is a methyl group and X is a bond. This compound is known as 1-methoxy-2-propylamin; correspondingly compound of formula II is a compound wherein R₁ is a methyl group, R₂ is hydrogen and X is a bond.

Preferably the compound of formula I has a defined stereo isomerism. In particular it may be a (R) or (S) enantiomer or a defined mixture thereof, the chiralic carbon atom being the carbon atom to which the primary amino group is bonded. Particularly preferred is a pure (R) or (S) enantiomer.

Hence the compound of formula II should have the desired stereo isomerism of compound of formula I. It is an object of the invention that the stereo isomerism of the compound of formula II is kept and that all or at least the predominant amount of the obtained product of formula I has the same stereo isomerism.

In a most preferred embodiment the compound of formula I is (S)-1-methoxy-2-propylamin and the compound of formula II is (S)-alaninol.

To Process Step a)

In the first process step a) the primary amino group of the compound of formula II is protected and reacted with a protecting agent. Without such protection the hydrogen atoms of the primary amino group could be deprotonated as well in process step b) and in process step c) alkylation at the deprotonated nitrogen could occur.

The protecting agent should react with the primary amino group so that both hydrogen of the primary amino group are replaced either by a double bond or two single bonds to other atoms than hydrogen; such other atoms being in particular carbon or nitrogen.

Suitable protecting agents that undergo such reactions with are primary amino group are known.

In a preferred embodiment of the invention a protecting agent is selected which reacts with the primary amino group to give an imine group, respectively a Schiff base. Suitable protecting agents which undergo such reaction are compounds with at least one carbonyl group (for short carbonyl compound) which are preferably compounds with one aldehyde or one keto group.

Preferred examples of such protecting agents are benzaldehyde, derivatives of benzaldehyde, benzophenone and derivatives of benzophenone; such protecting agents react with the primary amino groups forming benzylidene and diphenyldimethylene derivatives.

Hence—in a preferred embodiment—an imine group is formed by reacting the primary amino group with a carbonyl compound as protection agent in step a).

Preferably, the carbonyl compound has no further functional groups besides the carbonyl group. Preferably, the carbonyl compound has a low molecular weight, for example a molecular weight below 1000 g/mol, in particular below 500 g/mol, more preferred below 200 g/mol. Preferred carbonyl compounds have an aldehyde group. Suitable carbonyl compounds are, for example, benzaldehyde or p-methoxybenzaldehyde, p-methylbenzaldehyde, o-nitrobenzaldehyde or p-nitrobenzaldehyde.

The protecting agent may be used in molar excess relative to the amino alcohol to ensure that all primary amino groups are protected. In a preferred embodiment 1 to 1.5 mol of protecting agent is used on 1 mol amino alcohol.

The reaction of step a) may be performed in a solvent. Suitable solvents are, for example C5- to C16-alkanes like hexane, cyclohexane, heptane, aromatic solvents including alkyl- and alkoxy-aromatics and halogenated solvents like benzene, toluene, xylene (o-, p-, m-xylene or mixture of isomers), cumene, anisol, chlorobenzene and tert.-butylbenzene, cyclic or acyclic ethers like diethylether, diisopropylether, tert.-butyldimethylether (MTBE), tert.-butylethylether, trimethylorthoformate, tetrahydrofuran (THF) or dioxane, or ethyleneoxide ethers, in particular glymes like 1,2-dimethoxyethane (monoglyme), diethyleneglycoldimethylether (diglyme), 1,2-diethoxyethane (ethylglyme), diethoxy-diethylene glycol (ethyl diglyme), diethylene glycol dibutylether (butyl diglyme) et al. or polyethers like poly(ethylene glycol) dimethylether et al. or aliphatic halogenated solvents like dichloromethane, dichloroethane, trichloromethane and its mixtures.

It is advantageous for the reaction and its conversion to perform azeotropic removal of water using a suitable solvent.

The reaction may be performed at elevated temperatures, for example between 10 and 150° C. The reaction may be performed under reduced, normal or excess pressure, appropriately normal pressure.

The obtained solution comprises the protected amino alcohol.

To Process Step b)

In process step b) the alcohol (hydroxyl) group of the protected amino alcohol is deprotonated by a deprotonating agent.

Suitable deprotonating agents are for example a metal, a metal hydride, a metal alcoholate or any other suitable basic compound, like an amine.

Preferred deprotonating agents are a metal, a metal hydride or a metal alcoholate; whereby the metal (either as pure element or as cation of the listed compounds) may preferably be any metal of any of groups I to III of the periodic system. The term “alcoholate” shall be any compound with a hydroxyl group wherein the hydroxyl group is deprotonated.

Particularly preferred is an alkali or earth alkali metal, an alkali or earth alkali hydride or an alkali or earth alkali alcoholate.

More preferred are metal alcoholates, in particular alkali metal alcoholates or earth alkali metal alcoholates, most preferred are alkali metal alcoholates.

Preferred metal alcoholates are those of hydrocarbons with one hydroxyl group, in particular a metal C1- to C10-alkylate, respectively a metal C1- to C4-alkylate as, for example, sodium methylate, sodium ethylate, potassium methylate, potassium ethylate.

Most preferred deprotonating agents are for example lithium C1- to C4-alkylates, sodium C1- to C4-alkylates or potassium C1- to C4-alkylates, in particular sodium methylate, sodium ethylate, potassium methylate or potassium ethylate.

Process step b) may be performed in presence of a solvent.

Suitable solvents are, for example, aliphatic solvents, for example C5- to C16-alkanes like hexane, cyclohexane, heptane, aromatic solvents, including alkyl & alkoxyaromatics, like toluene or anisol, ether including cyclic ethers, e.g. dioxane, tetrahydrofurane, or ethyleneoxide ethers, in particular glymes like 1,2-dimethoxyethane (monoglyme), diethyleneglycoldimethylether (diglyme), 1,2-diethoxyethane (ethylglyme), diethoxy-diethylene glycol (ethyl diglyme), diethylene glycol dibutylether (butyl diglyme) et al. or polyethers like poly(ethylene glycol) dimethylether et al.

Preferred solvents are hydrophobic solvents, in particular aliphatic or aromatic hydrocarbons or ethers, as for example C7- to C15 alkanes, alkylaromatic solvents or ethyleneoxide ethers.

More preferred are aromatic solvents, in particular aromatic hydrocarbons like toluene or xylene.

Preferably a mixture of the protected amino alcohol and a solvent is used in process step b).

Preferably, the protected amino alcohol, respectively the mixture, should be free of water and other protic solvents.

A metal as deprotonating agent may be used in solid or liquid (molten) form. The metal may be added to the protected amino alcohol, the solvent or the mixture thereof.

Other liquid or solid deprotonating agents may simply be given to the protected amino alcohol, or to the mixture of the solvent and the protected amino alcohol. In case of solid deprotonating agents other than metal a solvent should be selected which solves the solid deprotonating agent.

Preferably the deprotonating agent should be consumed in process step b). Hence, in a preferred embodiment the protected amino alcohol and the deprotonating agent are used in equimolar amounts or—even more preferred—the protected amino alcohol is used in excess. The ratio of equivalents deprotonating agent to protected amino alcohol could for example be from 1:1 to 1:2, in particular from 1:1.05 to 1:1.5.

Process step b) is performed at a temperature of at maximum 120° C. Preferably process step b) is performed at a temperature of at maximum 100° C., in particular of at maximum 80° C.

A very suitable range of temperature for step b) is from 0 to 80° C., in particular from 0 to 70° C. In a more preferred embodiment the temperature in step b) is kept from 0 to 70, respectively 10 to 60° C.

Process step b) may be performed at normal, at reduced or at increased pressure. Usually process step b) will be performed at a pressure of 0.4 to 3 bars, in particular at 0.8 to 1.5 bar and preferably simply at normal pressure (1 bar).

To Process Step c)

In process step c) the anion obtained in step b) is alkylated with an alkylation agent to give the corresponding ether.

The reaction under step c) is preferably started when all deprotonating agent in process step b) is consumed.

Suitable alkylating agents are well known. Usual alkylating agents correspond to the general formula III

(Alkyl)ₘ-Z,

wherein Alkyl is an alkyl group, preferably an alkyl group with 1 to 4 carbon atoms, most preferred a methyl group, m may be 1, 2 or 3 and Z is a one, two or three valent inorganic or organic, corresponding the actual meaning of n.

In particular n is 1 or 2.

In particular Z is a halogen, for example chloride, or an organic or inorganic ester group.

As examples alkyl chloride, alkyl mesylate, alkyl tosylate, dialkyl sulfate or dialkyl carbonate, trialkyl phosphate may be named.

As R₃ preferably is a methyl group, the preferred reaction is a methylating reaction using, for example, methyl chloride, dimethyl sulfate or dimethyl carbonate as methylating agent.

In one preferred embodiment the alkylating agent, respectively methylating agent, is used as a gas. The boiling point of methyl chloride is −23.8° C. at 1 bar.

In a preferred embodiment of step b) a solvent has been used. This solvent is preferably not removed in or before step c). Hence both, process step b) and c) are preferably performed in presence of the same solvent, which is most preferred an aromatic solvent, in particular toluene or xylene.

In reaction step c) the reactants may be used in equimolar amounts or either reactant may be used in excess.

In particular the ratio of equivalents alkylating agent to the equivalents of the deprotonating agent used in process step b) may for example be from 0.5:1 to 1:0.5.

Preferably the ratio of equivalents alkylating agent to the equivalents metal is from 0.5:1 to 0.99:1, even more preferred is a ratio from 0.9:1 to 0.99: 1.

Process step c) is performed at a temperature of at maximum 80° C. Preferably process step c) is performed at a temperature of at maximum 60° C., in particular of at maximum 40° C. A very suitable range of temperature for step c) is from 0 to 80° C., in particular from 0 to 60° C. In a more preferred embodiment the temperature in step c) is kept from 10 to 40° C.

Process step c) may be performed at normal, at reduced or at increased pressure. Usually process step c) will be performed at a pressure of 0.4 to 3 bars, in particular at 0.8 to 1.5 bar and preferably simply at normal pressure (1 bar).

To Process Step d)

Finally the protecting group is removed in process step d) giving the ether of formula I.

Processes for such removal are well known to the man skilled in the art. Removal might be done by hydrogenation (S. Hecht et al. (J. Am. Chem. Soc. 2012, 134, 8718.) or by acid hydrolysis.

In a preferred embodiment removal is done by acid hydrolysis.

Acid hydrolysis can be performed as known to the man skilled in the art in analogy to known ester hydrolyses in the presence of catalytic or stoichiometric amounts of acid and water. Commonly, a mixture of water and an aprotic organic solvent is used. Examples for acids are halogen hydracids, sulfuric acid, organic sulfonic acids like p-toluenesulfonic acid, methanesulfonic acid, phosphoric acid or acidic ion exchange resins.

The following figure shows the sequence for all process steps a) to d) for (S)-alaninol as amino alcohol:

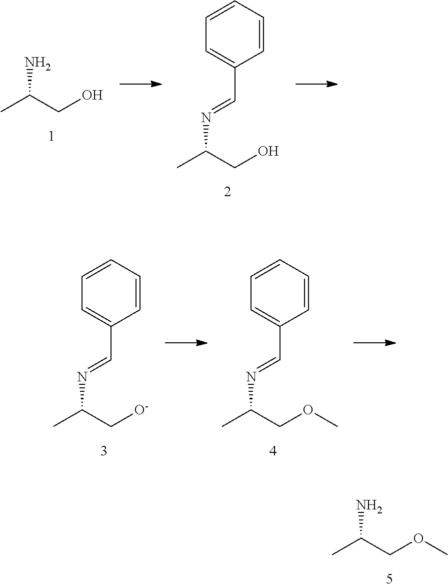

Preferably all steps a) to d) are made in the same solvent without separation of the intermediate products (one pot synthesis).

Further Work Up

The process comprising steps a) to d) usually results in a suspension or solution comprising the ether of formula I, salts that have been formed, for example sodium chloride, organic solvent that has been used and further by-products.

First, the pH value of the suspension or solution may be adjusted to have ether of formula I available at a neutral form and not as a salt. Thereafter remaining solids may be removed by filtration. Such pH adjustments and filtration would usually be done before any distillation.

The ether of formula I may be distilled from such suspension or solution after the pH value has been adjusted to have ether of formula I available at a neutral form and may be further purified thereafter, for example by further distillation.

Alternatively the salts may be removed after the distillation of the ether by adding an amount of water which is sufficient to solve any salts. The obtained aqueous salt solution and the solvent form distinct phases and can be separated easily.

When the deprotection step d) is performed under aqueous acidic conditions, recovery of the protecting agent and the organic solvent are possible by phase separation of the aqueous phase including the ether of formula I in protonated form. Further work-up includes adjustment of the pH of the aqueous phase to generate the ether of formula I in neutral form and subsequent removal of water by distillation. Preferentially, an organic solvent e.g. toluene is added to azeotropically remove water. After removal of water the ether of formula I can be fine-distilled by addition of a sump thinner.

By-products that may have been formed are compounds with substitution at the nitrogen atoms, in particular compounds wherein the nitrogen atom is alkylated as well, either once (giving a secondary amino group) or even twice (giving a tertiary amino group).

It is advantage of the claimed process that such by-products are not or at least hardly formed.

In particular the molar ratio of the ether of formula I to the by-product which is N-mono alkylated as well is usually more than 99:1, preferably more than 999:1.

The selectivity of the ether of formula I is usually more than 95%, in particular more than 97, respectively more than 99%, such selectivity being the ratio of the equivalents of the ether of formula I to the sum of the equivalents of all and any alkylated compounds×100%.

It is a further advantage of the claimed process that the stereo isomerism of the amino alcohol is kept. Usually at least 90%, typically at least 95%, respectively at least 98% of the ether of formula I obtained by the process has the same stereoisomerism as the amino alcohol. Hence starting with (S)-alaninol will give by methylating at least 90%, typically at least 95%, respectively at least 98% (S)-1-methoxy-2-propylamine.

The process allows high yields of ether.

Furthermore the process claimed is a very effective and efficient process which can be performed as one pot synthesis of at least steps b) and c). In particular process steps b), c) and d) or all process steps a) to d) may be performed as one pot synthesis. Such one pot synthesis is preferably performed without isolation of intermediates and with the use of one solvent, only, if any.

### EXAMPLES

MOIPA shall be any 1-methoxy-2-propylamin

(S)-MOIPA shall be the stereo isomer (S)-1-methoxy-2-propylamin.

N-Me-MOIPA shall be (S)-1-methoxy-2-propylamin with an additional methylation at the nitrogen atom ((2S)-1methoxy-N-methyl-propan-2-amine (IUPAC-name).

Gas chromatography (Hewlett Packard 6890 N with FID detector; column 1: 25 m Hydrodex-γ-TBDAc, inner diameter 0.25 mm, outer diameter 0.40 mm, film diameter 0.25 μm, oven program: temperature start: 60° C., hold: 0 min, in 5 K/min steps to temperature end: 220° C., hold: 1 min; column 2: 30 m Optima 5 MS, inner diameter 0.25 mm, outer diameter 0.45 mm, film diameter 1.0 μm, oven program: temperature start: 60° C., hold: 5 min, in 15 K /min steps to temperature end: 280° C., hold: 1 min.column 3: 50 m Optima 5 Accent, inner diameter 0.20 mm, outer diameter 0.45 mm, film diameter 0.35 μm, oven program: temperature start: 60° C., hold: 5 min, in 15 K/min steps to temperature end: 300° C., hold: 1 min.) was used to determine the yield and selectivity. The yield and selectivity were calculated from the area of the peaks of the gas chromatogram using calibration methods.

### Example 1

30 g of benzylidene-alaninol (0.18 mol, resulting from protecting alaninol with benzaldehyde) and 120 g of xylenes were brought to 60° C. During 30 min 33.07 g of sodium methylate, 30% in methanol (0.18 mol) were added dropwise while methanol was distilled off at 250 mbar (temperature of reaction mixture=57° C., reflux ratio 1:1, 10 cm column filled with 3*3 mm V2A rings, temperature at the top of distillation column=35° C.). After addition of sodium methylate was finished methanol was further distilled off at 250−70 mbar and simultaneously 69 g of xylenes were added.

The orange suspension was brought to 20 to 25° C. During 30 min 23.39 g of dimethylsulfate (0.18 mol) were added while cooling with an ice bath to maintain the temperature at 20 to 25° C. The orange thick suspension was stirred overnight. Then, 150 mL of distilled water were added and the reaction mixture was stirred for further 30 min. The phases were separated and the aqueous phase was extracted with 100 mL xylenes. The combined organic phases were dried over Na₂SO₄ and the solvent was removed under reduced pressure (30−5 mbar) at 65° C. to yield Benzylidene-(S)-MOIPA as an oil in 80% yield and in 89 GC-a % purity.

### Example 2

62.08 g of Benzylidene-Alaninol (0.38 mol) and 215 g of xylenes were brought to 130° C. During 30 min 68.43 g of sodium methylate, 30% in methanol (0.38 mol) were added dropwise while methanol was distilled off at normal pressure (temperature of the reaction mixture=110° C., reflux ratio 5:1, 10 cm column filled with 3*3 mm V2A rings, temperature at the top of the distillation column=68° C.). After addition of sodium methylate was finished 90 mL of methanol and xylenes was further distilled off and simultaneously 30 mL of xylenes were added.

The orange suspension was brought to 20-25° C. and stirred overnight. Then, the reaction mixture was brought to 100° C. and during 30 min 48.41 g of dimethylsulfate (0.38 mol) were added while the temperature was kept at 120° C. and then stirring was continued at 100 to 120° C. for 2 h. The black reaction mixture was brought to room temperature and 200 mL distilled water were added and the reaction mixture was stirred for further 30 min. The phases were separated and the organic phase was dried over Na₂SO₄ and the solvent was removed under reduced pressure (30−15 mbar) at 65° C. to yield Benzylidene-(S)-MOIPA as a black viscous oil in 3% yield and in 5.3 GC-a % purity.

1: A process for preparing an ether of formula I 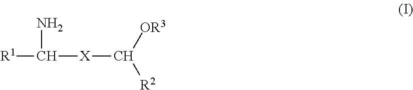
from an amino alcohol of formula II 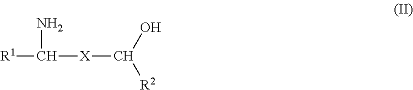
where R₁ and R₂ are each independently hydrogen or an alkyl group with 1 to 10 C atoms, R₃ is an alkyl group with 1 to 10 carbon atoms and X is a bond or a hydrocarbon group with 1 to 10 carbon atoms,
the process comprising:
a) protecting the primary amino group in formula II with a protecting agent, to obtain a protected amino alcohol;
b) deprotonating the protected amino alcohol obtained in a) with a deprotonating agent, to obtain an anion;
c) alkylating the anion obtained in b) with an alkylation agent to give a corresponding ether; and
d) removing the protecting group to obtain the ether of formula I,
wherein b) is performed at a temperature of at maximum 120° C. and c) is performed at a temperature of at maximum 80° C.

2: The process of claim 1, wherein R₁ is hydrogen or an alkyl group with 1 to 4 C atoms, R₂ is hydrogen, R₃ is an alkyl group with 1 to 4 C atoms and X is a bond or an alkylene group with 1 to 10 carbon atoms.

3: The process of claim 1, wherein R₁ is a methyl group, R₂ is hydrogen, R₃ is a methyl group and X is a bond.

4: The process of claim 1, wherein the compound of formula II is a pure (R) or (S) enantiomer.

5: The process of claim 1, wherein in a) an imine group is formed by reacting the primary amino group with a carbonyl compound as the protection agent.

6: The process of claim 1, wherein the deprotonating agent in b) is an alkali or earth alkali alcoholate.

7: The process of claim 1, wherein the alkylation agent in c) is selected from the group consisting of alkyl chloride, dialkyl sulfate, and dialkyl carbonate.

8: The process of claim 1, wherein b) and c) are performed at a temperature from 0 to 80° C.

9: The process of claim 1, wherein b) and c) are performed in the presence of a solvent.

10: The process of claim 1, wherein a) to d) are performed in the same solvent without separation of intermediate products.
